(19) United States
(12) Patent Application Publication
(10) Pub. No.: US 2019/0183982 A1
(43) Pub. Date: Jun. 20, 2019

(54) POLYMERIC RED BLOOD CELL-LIKE PARTICLES

(71) Applicant: Milwaukee School of Engineering, Milwaukee, WI (US)

(72) Inventors: Wujie Zhang, Milwaukee, WI (US); Rebecca Ann Schroeder, Lena, WI (US); Sydney Jeanene Stephens, Chesterfield, MO (US); Haley Eleanor Stephens, Wind Lake, WI (US); Kellen Daniel O'Connell, Green Bay, WI (US); Nataline Marie Duerig, Darien, WI (US); Devon McCune, Milwaukee, WI (US); Jung Chull Lee, Brookfield, WI (US); Gene A. Wright, Pewaukee, WI (US)

(21) Appl. No.: 16/224,197

(22) Filed: Dec. 18, 2018

Related U.S. Application Data
(60) Provisional application No. 62/607,202, filed on Dec. 18, 2017.

(51) Int. Cl. A61K 38/42; A61K 9/00; C07K 14/805; A61K 31/732; A61K 31/722

(52) U.S. Cl. CPC A61K 38/42; A61K 9/0026; A61K 31/722; A61K 31/732; C07K 14/805

(57) ABSTRACT

Disclosed herein are synthetic particles that are shaped like red blood cells. The particles include pectin and oligochitosan and optionally a bioactive agent. In addition, methods of making the synthetic particles via electrospray techniques are provided.

### CROSS-REFERENCE TO RELATED APPLICATIONS

This application is a non-provisional of and claims the benefit of U.S. Provisional Patent Application No. 62/607,202, filed Dec. 18, 2017, which is incorporated herein by reference in its entirety.

### BACKGROUND

According to the American Red Cross, in the United States someone is in need of blood every two seconds. In addition, with Zika virus, HIV, and Hepatitis scares, the Food and Drug Administration requires 11-12 different blood screening tests. The growing global need for a consistent blood supply needs to be addressed as the demand continues to increase and the number of eligible donors decreases.

### SUMMARY

A potential solution to the blood-shortage problem is to develop oxygen therapeutics through carrier size reduction and functionalization of pectin-oligochitosan hydrogel particles.

In some aspects, disclosed are synthetic particles comprising pectin; and oligochitosan, wherein the synthetic particle has a biconcave discoid shape, and a largest linear dimension of about 4 μm to about 12 μm.

In some aspects, disclosed are methods of making a synthetic particle having a shape of a red blood cell, the method comprising electrospraying a pectin solution comprising pectin, a viscosity enhancer, a solution modifier and a first solvent into an oligochitosan solution comprising oligochitosan and a second solvent to provide a particle suspension comprising a synthetic particle as disclosed herein.

### BRIEF DESCRIPTION OF THE DRAWINGS

FIG. 1 is a photograph of an exemplary electrospray setup.

FIGS. 2A-C are plots showing single-variable relationships with the size of exemplary particles. FIG. 2A is a plot showing the relationship between voltage and particle size;

FIG. 2B is a plot showing the relationship between flow rate and particle size; and FIG. 2C is a plot showing the relationship between flow height and particle size.

FIGS. 3A-C are plots showing two-variable relationships with the size of exemplary particles. FIG. 3A is a plot showing the relationship between voltage, height and particle size;

FIG. 3B is a plot showing the relationship between flow rate, voltage and particle size; and FIG. 3C is a plot showing the relationship between height, flow rate and particle size.

FIG. 4 is a set of microscope images showing that the disclosed methods provide smaller particles compared to previously known methods. Left image: particle with average diameter of about 300 μm; Right image: exemplary particles with an average diameter of about 7 μm.

FIG. 5 is a microscope image showing particles following treatment with 150 mM CaCl₂.

FIG. 6 is a microscope image showing the stability treatment of particles with glutaraldehyde.

### DETAILED DESCRIPTION

Disclosed herein are synthetic particles that are shaped like red-blood cells having a biconcave discoid shape. The synthetic particles are made by electrospraying techniques through the use of pectin and oligochitosan. The disclosed electrospraying methods were able to achieve red-blood cell shaped particles having an average diameter of less than 10 μm. This is a significant improvement over previously made particles in the art that have an average diameter of no less than 100 μm. Accordingly, the disclosed synthetic particles can be used in biomedical applications, such as being injected intravenously for drug delivery and/or imaging applications, where the particles of the art would be unusable.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “an” and “the” include plural references unless the context clearly dictates otherwise. The present disclosure also contemplates other embodiments “comprising,” “consisting of” and “consisting essentially of,” the embodiments or elements presented herein, whether explicitly set forth or not.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Other meanings of “about” may be apparent from the context, such as rounding off, so, for example “about 1” may also mean from 0.5 to 1.4.

As used herein, the term “imaging agent,” refers to a molecule or compound that can be detected directly or after applying a stimulus. Examples of imaging agents include luminescent labels which emit radiation on exposure to an external source of radiation or other stimulus, e.g. fluorescent materials or fluorophores (which emit light when exposed to light), chemiluminescent materials (which emit light during chemical reaction), electroluminescent materials (which emit light on application of an electric current), phosphorescent materials (in which emission of light continues after exposure to light stimulus has ended) and thermoluminescent materials (which emit light once a certain temperature is exceeded). Examples of fluorophores include fluoresceins, xanthenes, cyanines, naphthalenes, coumarins, oxadiazoles, pyrenes, oxazines, acridines, arylmethines, Alexa Fluors and tetrapyrroles. Further fluorophores include quantum dots, which emit highly specific wavelengths of electromagnetic radiation after stimulation, for example by electricity or light.

Other imaging agents include radioactive labels, including positron emitting nuclei such as ¹⁸F, ⁶⁴Cu or ¹²⁴I which can be detected by imaging techniques such as positron emission topography (PET). Other radioactive labels such as ¹⁴C, ³H, or iodine isotopes such as ¹²³I and ¹³¹I, which can be detected using autoradiographic analysis or scintillation detection for example, can also be used. In the case of gamma-emitting nuclei, imaging techniques such as single photon emission computed tomography (SPECT) can be used. Other imaging agents include those that are NMR-active, which can be detected by magnetic resonance techniques, for example magnetic resonance imaging (MRI) or nuclear magnetic resonance (NMR) detectors, the agents typically comprising one or more NMR-active nuclei that are not generally found in concentrated form elsewhere in the organism or biological sample, examples being ¹³C, ²H (deuterium) or ¹⁹F. Further imaging agents include those comprising atoms with large nuclei, for example atoms with atomic number of 35 or more, preferably 40 or more and even more preferably 50 or more, for example iodine or barium, which are effective contrast agents for X-ray photographic techniques or computed tomography (CT) imaging techniques.

As used herein, the term “shelf-life” refers to the synthetic particle being able to maintain its structural (e.g., size) and/or functional features for a specified amount of time, e.g., while being stored. For example, the synthetic particle being able to maintain its average particle size within ±2 μm for 20 days at storage conditions corresponds to the particle having a shelf-life of greater than or equal to 20 days. In addition, shelf-life can be described as the synthetic particle being able to maintain the bioactivity/therapeutic effect (within, e.g., ±5% of its original activity) of a bioactive agent after a specified amount time in storage conditions relative to its bioactivity/therapeutic effect when it is first encapsulated and/or adhered to the particle.

As used herein, the term “therapeutic agent” refers to an agent capable of treating and/or ameliorating a condition or disease, or one or more symptoms thereof, in a subject. Examples include hemoglobin, artificial oxygen transporters, immune stimulants, blood clotting inhibitors and/or inducers, nanoparticles, and lipophilic molecules.

### 2. Synthetic Particles

Disclosed herein are synthetic particles that can mimic structural and functional features of red blood cells. The particles include pectin and oligochitosan. The synthetic particles may further include a divalent cation, covalent cross-linking agent, or both. In addition, the particles may include a bioactive agent.

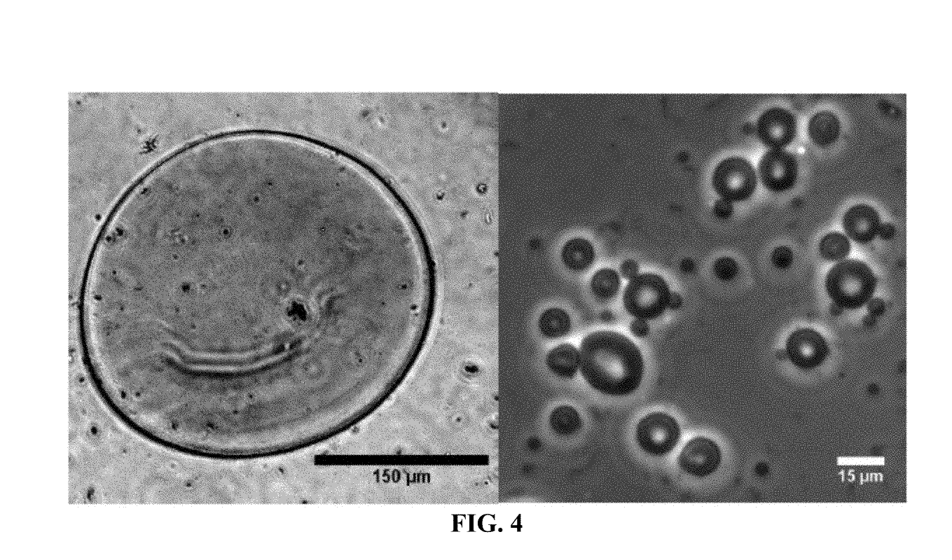

The disclosed synthetic particles have a biconcave discoid shape (see FIG. 4—where the particles have a disk shape with two generally concave central depressions). In addition, the synthetic particles may have a largest linear dimension of about 4 μm to about 12 μm, such as about 4 μm to about 10 μm or about 5 μm to about 9 μm. In some embodiments, the synthetic particle has a largest linear dimension of about 7 μm. The dimension can be measured across the largest portion of the particle that corresponds to the parameter being measured. For example, the linear dimension being measured can be the diameter of the particle. The linear dimension may be measured by light microscopy and/or electron microscopy techniques (e.g., transmission electron microscopy, scanning electron microscopy, etc.).

In some embodiments, the synthetic particle has a largest linear dimension of less than or equal to 12 μm, less than or equal to 11 μm, less than or equal to 10 μm, less than or equal to 9 μm, or less than or equal to 8 μm. In some embodiments, the synthetic particle has a largest linear dimension of greater than or equal to 2 μm, greater than or equal to 3 μm, greater than or equal to 4 μm, greater than or equal to 5 μm, or greater than or equal to 6 μm.

The synthetic particles may also be described by its average particle diameter as measured by, e.g., dynamic light scattering techniques. For example, the synthetic particle may have an average diameter of about 2 μm to about 20 μm as measured by dynamic light scattering, such as about 3 μm to about 18 μm or about 5 μm to about 15 μm as measured by dynamic light scattering. In some embodiments, the synthetic particle has an average diameter of greater than or equal to 2 μm, greater than or equal to 2.5 μm, greater than or equal to 3 μm, greater than or equal to 3.5 μm, or greater than or equal to 4 μm as measured by dynamic light scattering. In some embodiments, the synthetic particle has an average diameter of less than or equal to 20 μm, less than or equal to 18 μm, less than or equal to 16 μm, less than or equal to 14 μm, or less than or equal to 12 μm as measured by dynamic light scattering.

The disclosed synthetic particles having a biconcave discoid shape have increased surface area relative to a spherical particle of similar dimensions, and this may be advantageous for applications such as drug delivery (e.g., treatment of disease) and molecular imaging (e.g., diagnosis of disease). The synthetic particles may have a surface area of about 45 μm² to about 300 μm², such as about 50 μm² to about 275 μm² or about 60 μm² to about 250 μm². In some embodiments, the synthetic particle has a surface area of less than 300 μm², less than 275 μm², less than 250 μm², less than 225 μm², less than 200 μm², less than 175 μm², or less than 150 μm². In some embodiments, the synthetic particle has a surface area of greater than 45 μm², greater than 50 μm², greater than 75 μm², greater than 100 μm², greater than 125 μm², or greater than 150 μm².

In addition, the synthetic particle may have a volume that is advantageous for applications such as drug delivery and molecular imaging. The synthetic particle may have a volume of about 20 μm³ to about 315 μm³, such as about 35 μm³ to about 300 μm³ or about 50 μm³ to about 275 μm³. In some embodiments, the synthetic particle has a volume of less than 315 μm³, less than 300 μm³, less than 275 μm³, less than 250 μm³, less than 225 μm³, less than 200 μm³, less than 175 μm³, or less than 150 μm³. In some embodiments, the synthetic particle has a volume of greater than 20 μm³, greater than 35 μm³, greater than 50 μm³, greater than 75 μm³, greater than 100 μm³, greater than 125 μm³, or greater than 150 μm³.

The disclosed synthetic particles may mimic red blood cells not only by shape and size, but also by how the synthetic particle functions. For example, the synthetic particles may be able to reversibly deform, which can allow the synthetic particles to pass through, e.g., capillaries having a diameter of less than 3 μm.

The synthetic particle may also have advantageous stability. For example, the synthetic particle may have a shelf-life of greater than or equal to 10 days, greater than or equal to 15 days, greater than or equal to 20 days, greater than or equal to 25 days, greater than or equal to 30 days, greater than or equal to 35 days, greater than or equal to 40 days, greater than or equal to 42 days, greater than or equal to 45 days, or greater than or equal to 50 days.

The synthetic particle may be a hydrogel, which as used herein refers to a water-swollen polymeric material that maintains a distinct three-dimensional structure. In some embodiments, the synthetic particle is described as a hydrogel microcapsule.

A. Pectin

Pectin is a naturally occurring polymer of galacturonic acid with carboxyl groups, which can be found in citrus fruits such as apples. Pectin may have a degree of methyl esterification. For example, pectin may be a low methyl pectin (e.g., low methoxy pectin) having a low methyl esterification or may be high methyl pectin (e.g., high methoxy pectin) having a high methyl esterification. As used herein, low methyl esterification refers to pectin with less than 50% of the acid units esterified. In addition, as used herein, high methyl esterification refers to pectin with greater than 50% of the acid units esterified. In some embodiments, pectin is low methoxy pectin. In other embodiments, pectin includes both low methoxy pectin and high methoxy pectin. In some embodiments, pectin has a degree of esterification of about 20.4%.

Pectin may be present in the synthetic particle at varying amounts. For example, pectin may be present at about 2% to about 35% by weight of the synthetic particle, such as about 5% to about 30% or about 10% to about 25% by weight of the synthetic particle. In some embodiments, pectin may be present at about 2% to about 90% by weight of the synthetic particle, such as about 2% to about 90%, about 5% to about 90%, about 10% to about 90%, about 20% to about 90%, about 30% to about 90%, about 40% to about 90%, about 50% to about 90%, about 60% to about 90%, about 70% to about 90%, about 80% to about 90%, about 2% to about 75%, about 5% to about 75%, about 10% to about 75%, about 20% to about 75%, about 30% to about 75%, about 40% to about 75%, about 50% to about 75%, about 60% to about 75%, about 2% to about 50%, about 5% to about 50%, about 10% to about 50%, about 20% to about 50%, about 30% to about 50%, or about 40% to about 50% by weight of the synthetic particle. In some embodiments, pectin is present at greater than or equal to 2%, greater than or equal to 5%, greater than or equal to 10%, greater than or equal to 15%, greater than or equal to 20%, greater than or equal to 25%, greater than or equal to 30%, greater than or equal to 35%, greater than or equal to 40%, greater than or equal to 50%, greater than or equal to 60%, greater than or equal to 70%, greater than or equal to 80%, or greater than or equal to 90% by weight of the synthetic particle. In some embodiments, pectin is present at less than or equal to 90%, less than or equal to 80%, less than or equal to 70%, less than or equal to 60%, less than or equal to 50%, less than or equal to 40%, less than or equal to 35%, less than or equal to 30%, less than or equal to 25%, or less than or equal 20% by weight of the synthetic particle. In some embodiments, pectin is present at about 3.25% by weight of the synthetic particle.

B. Oligochitosan

Oligochitosan refers to low molecular weight chitosan, where chitosan is a polysaccharide composed of randomly distributed β-(1->4)-linked D-glucosamine (deacetylated unit) and N-acetyl-D-glucosamine (acetylated unit). Oligochitosan may have a molecular weight of about 0.5 kD to about 7 kD, such as about 1 kD to about 5 kD or about 1 kD to about 4.5 kD. In some embodiments, oligochitosan has a molecular weight of greater than or equal to 0.5 kD, greater than or equal to 1 kD, or greater than or equal to 2 kD. In some embodiments, oligochitosan has a molecular weight of less than or equal to 7 kD, less than or equal to 6 kD, less than or equal to 5 kD, or less than or equal to 4 kD. In some embodiments, oligochitosan is about 2 kD.

Oligochitosan may be present in the synthetic particle at varying amounts. For example, oligochitosan may be present at about 0.5% to about 10% by weight of the synthetic particle, such as about 1% to about 7% or about 1% to about 5% by weight of the synthetic particle. In some embodiments, oligochitosan is present at greater than or equal to 0.5%, greater than or equal to 1%, greater than or equal to 1.5%, greater than or equal to 2%, or greater than or equal to 2.5% by weight of the synthetic particle. In some embodiments, oligochitosan is present at less than or equal to 7%, less than or equal to 6%, less than or equal to 5%, or less than or equal 4.5% by weight of the synthetic particle. In some embodiments, oligochitosan is present at about 5% by weight of the synthetic particle.

C. Divalent Cation/Covalent Cross-linking Agent

The synthetic particle may include a divalent cation, a covalent cross-linking agent, or both. The divalent cation, covalent cross-linking agent, or both may be added to the synthetic particle after the particle has been formed, which is described in greater detail below. The divalent cation, covalent cross-linking agent or both may help stabilize the synthetic particle and may be advantageous to the overall shelf-life of the synthetic particle. Examples of the divalent cation include, but are not limited to, Ca²⁺, Mg²⁺, and compounds that include Ca²⁺ and Mg²⁺ where the divalent cation can be liberated from the compound and can interact with the synthetic particle under the appropriate conditions. In some embodiments, a compound including a divalent cation can be referred to as a divalent cation source. Examples of such compounds/divalent cation sources include, but are not limited to, CaCl₂, BaCl₂, CaCO₃, CaSO₄, and combinations thereof. In addition, an example of a covalent cross-linking agent includes, but is not limited to, glutaraldehyde. In some embodiments, the divalent cation is Ca²⁺. In some embodiments, the divalent cation and/or divalent cation source is CaCl₂.

D. Bioactive Agent

The synthetic particle may include a bioactive agent. Examples of bioactive agents include, but are not limited to, therapeutic agents and imaging agents. In some embodiments, the bioactive agent is selected from the group consisting of a therapeutic agent, an imaging agent, and a combination thereof. In some embodiments, the synthetic particle includes more than one bioactive agent, such as at least one therapeutic agent and at least one imaging agent; at least two therapeutic agents, at least two imaging agents, and combinations thereof. Examples of therapeutic agents include, but are not limited to, hemoglobin, hemoglobin-based oxygen carriers, and perfluorocarbon-based oxygen carriers. In addition, examples of imaging agents include, but are not limited to, stannous pyrophosphate and technetium 99. In some embodiments, the bioactive agent is hemoglobin.

The bioactive agent may be present in the synthetic particle at varying amounts. For example, the bioactive agent may be present at about 0.1% to about 5% by weight of the synthetic particle, such as about 0.1% to about 3% or about 1% to about 3% by weight of the synthetic particle. In some embodiments, the bioactive agent is present at greater than or equal to 0.1%, greater than or equal to 0.2%, greater than or equal to 0.3%, greater than or equal to 0.4%, or greater than or equal to 0.5% by weight of the synthetic particle. In some embodiments, the bioactive agent is present at less than or equal to 5%, less than or equal to 4%, less than or equal to 3%, or less than or equal to 2.5% by weight of the synthetic particle. In some embodiments, the bioactive agent is present at about 0.1% by weight of the synthetic particle.

The bioactive agent may be encapsulated within the synthetic particle, bound to the surface of the synthetic particle, or both. For example, the bioactive agent may be part of a solution used to provide the synthetic particle and encapsulated during the process of making. In addition, the synthetic particle may have the bioactive agent bound to the surface of the particle after the particle is provided. The bioactive agent may be conjugated to the surface of the synthetic particle by conjugation techniques known within the art using functional groups present on the pectin, oligochitosan or both. Further, in some embodiments, the bioactive agent may be localized to specific locations of the synthetic particle, e.g., an imaging agent localized to the surface of the synthetic particle and a therapeutic agent encapsulated within the synthetic particle.

In embodiments where the synthetic particle includes a therapeutic agent, the therapeutic agent may be released over a period of time from the synthetic particle. For example, the therapeutic agent may be released under a controlled release, may be released as a burst release, may be released due to interaction with an external stimulus, or combinations thereof.

### 3. Methods of Making the Synthetic Particles

Also disclosed herein are methods of making the synthetic particles having a shape of a red blood cell. The synthetic particles may be made by electrospray methods. For example, the method may include a pectin solution that is electrosprayed into an oligochitosan solution to provide a suspension comprising the synthetic particles as described above.

In particular, the method may include adding pectin, a viscosity enhancer and a solution modifier to a first solvent to provide a pectin solution. The first solvent may be an aqueous solvent, such as purified water. Pectin may be present in the pectin solution at about 1% to about 10% by weight/volume, such as about 1% to about 6%, about 1.5% to about 5%, or about 2% to about 4% by weight/volume. In some embodiments, pectin may be present in the pectin solution at about 1% to about 75% by weight/volume, such as about 1% to about 75%, about 1% to about 60%, about 1% to about 50%, about 1% to about 40%, about 1% to about 30%, about 1% to about 20%, about 1% to about 15%, about 1% to about 10%, about 5% to about 75%, about 5% to about 60%, about 5% to about 50%, about 5% to about 40%, about 5% to about 30%, about 5% to about 20%, about 5% to about 15%, about 5% to about 10%, about 10% to about 75%, about 10% to about 60%, about 10% to about 50%, about 10% to about 40%, about 10% to about 30%, about 10% to about 20%, about 10% to about 15%, about 20% to about 75%, about 20% to about 60%, about 20% to about 50%, about 20% to about 40%, about 20% to about 30%, about 30% to about 75%, about 30% to about 60%, about 30% to about 50%, or about 30% to about 40% by weight/volume. In some embodiments, pectin is present in the pectin solution at greater than or equal to 1%, greater than or equal to 1.5%, greater than or equal to 2%, greater than or equal to 2.5%, greater than or equal to 3%, greater than or equal to 3.5%, greater than or equal to 4%, greater than or equal to 5%, greater than or equal to 10%, greater than or equal to 15%, greater than or equal to 20%, greater than or equal to 25%, greater than or equal to 30%, greater than or equal to 40%, greater than or equal to 50%, or greater than or equal to 60% by weight/volume. In some embodiments, pectin is present in the pectin solution at less than or equal to 70%, less than or equal to 60%, less than or equal to 50%, less than or equal to 40%, less than or equal to 30%, less than or equal to 20%, less than or equal to 10%, less than or equal to 9%, less than or equal to 8%, less than or equal to 7%, less than or equal to 6%, or less than or equal to 5% by weight/volume. In some embodiments, pectin is present at about 3% to about 3.5% by weight/volume.

The viscosity enhancer and solution modifier may provide advantageous properties to the pectin solution that can aid in providing the disclosed synthetic particles via electrospraying techniques. The viscosity enhancer may include poly(ethylene oxide), poly(ethylene glycol), carboxylmethyl cellulose, or combinations thereof. In some embodiments, the viscosity enhancer is poly(ethylene oxide). The solution modifier may include glycerol. In some embodiments, the solution modifier is glycerol.

The viscosity enhancer may be present in the pectin solution at about 2% to about 5% by weight/volume, such as about 2.5% to about 4.5% or about 3% to about 5% by weight/volume. In some embodiments, the viscosity enhancer is present at greater than or equal to 2%, greater than or equal to 2.5%, or greater than or equal to 3% by weight/volume. In some embodiments, the viscosity enhancer is present in the pectin solution at less than or equal to 5%, less than or equal to 4.5%, or less than or equal to 4% by weight/volume. In some embodiments, the viscosity enhancer is present in the pectin solution at about 4% by weight/volume.

The solution modifier may be present in the pectin solution at about 1% to about 30% by weight/volume, such as about 2% to about 20% or about 3% to about 10% by weight/volume. In some embodiments, the solution modifier is present in the pectin solution at greater than or equal to 1%, greater than or equal to 3%, greater than or equal to 5%, greater than or equal to 10%, or greater than or equal to 15% by weight/volume. In some embodiments, the solution modifier is present in the pectin solution at less than or equal to 30%, less than or equal to 25%, less than or equal to 20%, or less than or equal to 15% by weight/volume. In some embodiments, the solution modifier is present in the pectin solution at about 5% by weight/volume.

In addition, a bioactive agent may be added to the first solvent, the pectin solution, or both. Description on the bioactive agent is discussed above. The bioactive agent may be present in the pectin solution at about 0.1% to about 10% by weight/volume, such as about 0.2% to about 8% or about 0.5% to about 7% by weight/volume. In some embodiments, the bioactive agent is present in the pectin solution at greater than or equal to 0.1%, greater than or equal to 0.2%, greater than or equal to 0.5%, or greater than or equal to 1% by weight/volume. In some embodiments, the bioactive agent is present in the pectin solution at less than or equal to 10%, less than or equal to 9%, less than or equal to 8%, or less than or equal to 7% by weight/volume.

The synthetic particles may encapsulate the bioactive agent at high efficiency, such as greater than 90% encapsulation efficiency, greater than 91% encapsulation efficiency, greater than 92% encapsulation efficiency, greater than 93% encapsulation efficiency, greater than 94% encapsulation efficiency, greater than 95% encapsulation efficiency, greater than 96% encapsulation efficiency, greater than 97% encapsulation efficiency, greater than 98% encapsulation efficiency, or greater than 99% encapsulation efficiency. In some embodiments, the synthetic particles encapsulate the bioactive agent at about 90% to about 99% encapsulation efficiency.

The method may also include adding oligochitosan to a second solvent to provide an oligochitosan solution. The second solvent may be an aqueous solvent, such as purified water. In some embodiments, the second solvent is the same as the first solvent. Oligochitosan may be present in the oligochitosan solution at about 1% to about 10% by weight/volume, such as about 2% to about 8% or about 3% to about 7% by weight/volume. In some embodiments, oligochitosan is present in the oligochitosan solution at greater than or equal to 1%, greater than or equal to 1.5%, greater than or equal to 2%, greater than or equal to 2.5%, or greater than or equal to 3% by weight/volume. In some embodiments, oligochitosan is present in the oligochitosan solution at less than or equal to 10%, less than or equal to 9.5%, less than or equal to 9%, less than or equal to 8.5%, or less than or equal to 8% by weight/volume. In some embodiments, oligochitosan is present in the oligochitosan solution at about 5% by weight/volume.

Electospraying the pectin solution into the oligochitosan solution may be performed under varying parameters to provide the disclosed synthetic particles. Such parameters include, but are not limited to, voltage, spray height, and flow rate. In particular, it has been found that the disclosed electrospraying parameters can provide significantly smaller red blood cell shaped synthetic particles compared to similar synthetic particles described in the art. For example, electrospraying may be performed at a voltage of about 5 kV to about 30 kV, such as about 8 kV to about 25 kV or about 10 kV to about 20 kV. The pectin solution may be electrosprayed at a height of about 5 cm to about 25 cm, such as about 10 cm to about 25 cm or about 12 cm to about 22 cm. In addition, the pectin solution may be electrosprayed at a flow rate of about 0.21 mL/h to about 15 mL/h, such as about 0.5 mL/h to about 12 mL/h or about 0.75 mL/h to about 8 mL/h.

After the particle suspension is provided, the synthetic particles may be isolated by using centrifugation, a filtration system or both.

The method may further include adding a solution that includes a divalent cation, a covalent cross-linking agent, or both to the particle suspension. The divalent cation and covalent cross-linking agent are described in further detail above. The divalent cation may be included in the solution at a concentration of about 0.1 mM to about 200 mM, such as about 1 mM to about 50 mM, about 0.1 mM to about 10 mM, or about 10 mM to about 150 mM. In addition, the covalent cross-linking agent may be included in the solution at a concentration of about 0.01 M to about 1 M, such as about 0.1 M to about 0.75 M or about 0.5 M to about 0.75 M.

### 4. Uses of the Synthetic Particles

The disclosed synthetic particles may be advantageous for a number of different applications. For example, the synthetic particles may be used in applications such as diagnostics, therapy, or both.

The synthetic particles may be used to deliver therapeutic agents, imaging agents, or both to a cell and/or subject. In some embodiments, the synthetic particles may be used in methods to treat a subject having a disease. Diseases may include, but are not limited to, cancer, blood disorders, and inflammatory disorders. In some embodiments, the disclosed synthetic particles may be used in blood transfusion methods, such as treating a subject that is in need of blood supplementation. By “treatment” it is meant that at least an amelioration of the symptoms associated with the condition afflicting the subject is achieved, where amelioration is used in a broad sense to refer to at least a reduction in the magnitude of a parameter, e.g., symptom, associated with the condition being treated. As such, treatment also includes situations where the pathological condition, or at least symptoms associated therewith, are completely inhibited, e.g., prevented from happening, or stopped, e.g., terminated, such that the subject no longer suffers from the condition, or at least the symptoms that characterize the condition. Thus, treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease; and/or (iii) relief, that is, causing the regression of clinical symptoms.

The subject to be treated can be one that is in need of therapy, where the subject to be treated is one amenable to treatment using the disclosed particles. Accordingly, a variety of subjects may be amenable to treatment using the particles disclosed herein. Generally, such subjects are “mammals”, with humans being of interest. Other subjects can include domestic pets (e.g., dogs and cats), livestock (e.g., cows, pigs, goats, horses, and the like), rodents (e.g., mice, guinea pigs, and rats, e.g., as in animal models of disease), as well as non-human primates (e.g., chimpanzees, and monkeys).

The amount of particles administered to a subject (for diagnosis and/or treatment) can be initially determined based on guidance of a dose and/or dosage regimen of the parent drug. In general, the synthetic particles can provide for targeted delivery, thus providing for at least one of reduced dose or reduced administrations in a dosage regimen. In addition, the particles may provide for extended release of the therapeutic.

The synthetic particles of the present disclosure can be delivered by any suitable means (e.g., pharmaceutical formulation), including oral, parenteral and topical methods. For example, pharmaceutical formulations can be formulated as applicator sticks, solutions, suspensions, emulsions, gels, creams, ointments, pastes, jellies, paints, powders, and aerosols. The pharmaceutical formulation may be provided in unit dosage form. In such form the pharmaceutical formulation may be subdivided into unit doses containing appropriate quantities of the particles of the present disclosure. The unit dosage form can be a packaged preparation, the package containing discrete quantities of the preparation, such as packeted tablets, capsules, and powders in pouches, vials or ampoules. Also, the unit dosage form can be a capsule, tablet, dragee, cachet, or lozenge, or it can be the appropriate number of any of these in packaged form.

Synthetic particles of the present disclosure can be present in any suitable amount, and can depend on various factors including, but not limited to, weight and age of the subject, state of the disease, etc. Suitable dosage ranges for the particles of the present disclosure include from 0.1 mg to 10,000 mg, or 1 mg to 1000 mg, or 10 mg to 750 mg, or 25 mg to 500 mg, or 50 mg to 250 mg. For instance, suitable dosages for the particles of the present disclosure include 1 mg, 5 mg, 10 mg, 20 mg, 30 mg, 40 mg, 50 mg, 60 mg, 70 mg, 80 mg, 90 mg, 100 mg, 150 mg, 200 mg, 250 mg, 300 mg, 350 mg, 400 mg, 450 mg, 500 mg, 550 mg, 600 mg, 650 mg, 700 mg, 750 mg, 800 mg, 850 mg, 900 mg, 950 mg or 1000 mg.

The synthetic particles of the present disclosure can be administered at any suitable frequency, interval and duration. The frequency of administration can vary depending on any of a variety of factors, e.g., severity of the symptoms, condition of the subject, etc. For example, the particles can be administered once an hour, or two, three or more times an hour, once a day, or two, three, or more times per day, or once every 2 days, 3 days, 4 days, 5 days, 6 days, or 7 days, so as to provide the desired dosage level to the subject. When the particles are administered more than once a day, representative intervals include 5 min, 10 min, 15 min, 20 min, 30 min, 45 min and 60 minutes, as well as 1 hr, 2 hr, 4 hr, 6 hr, 8 hr, 10 hr, 12 hr, 16 hr, 20 hr, and 24 hours. The particles of the present disclosure can be administered once, twice, or three or more times, for an hour, for 1 to 6 hours, for 1 to 12 hours, for 1 to 24 hours, for 6 to 12 hours, for 12 to 24 hours, for a single day, for 1 to 7 days, for a single week, for 1 to 4 weeks, for a month, for 1 to 12 months, for a year or more, or even indefinitely.

### 5. Examples

### Example 1—Synthesis and Characterization of Red Blood Cell-Like Particles

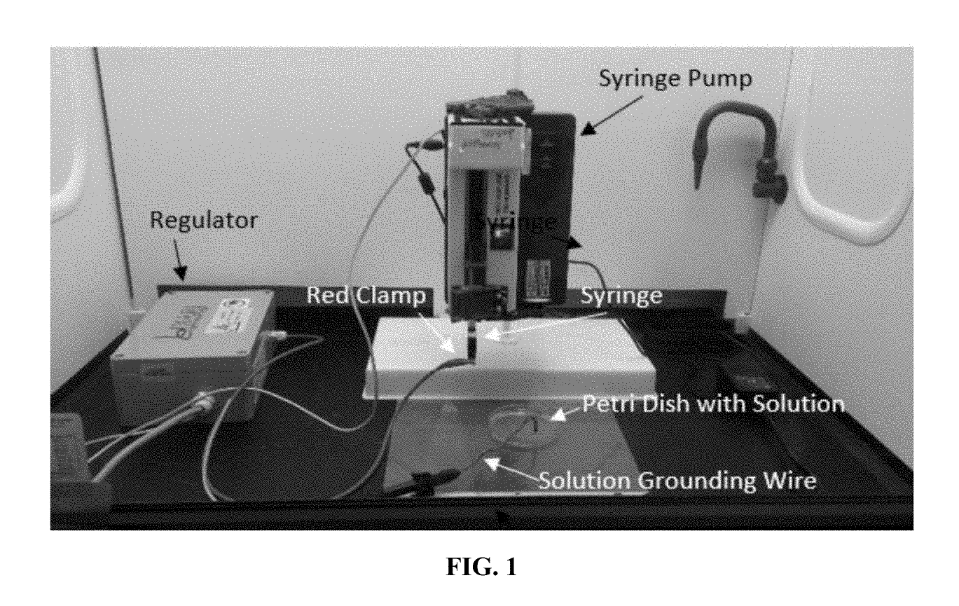

The artificial red blood cell development process includes various subsystems: size reduction, functionality, and stability. The parameters manipulated in order to achieve size reduction include voltage, flow rate, and height. The functionality of the artificial red blood cells were tested which includes testing the hemoglobin encapsulation efficiency. Finally, the stability of the produced capsules was tested by investigating the degradation of the capsules by adjusting the calcium chloride treatment concentrations. This example used electrospraying techniques, which an example set-up is shown in FIG. 1.

### Methods for Encapsulation Studies

Subsystem Specifications: Parameters for encapsulator: Voltage: 25 kV; Pressure: 600 mbar; Frequency: 2000 Hz; Amplitude: 6; Nozzle: 300 microns. Parameters for spectrometer: Wavelength: 410 nm.

Test Procedures for Encapsulation of Hemoglobin: The following steps were performed: 1. Prepare hemoglobin stock solutions of 2 mg/mL and 0.1 mg/mL hemoglobin in pectin. Add 7 mL of hemoglobin dissolved in water to 100 mL of 3.5% pectin solution. 2. At the given parameters, use a stopwatch to determine the flow rate of the encapsulator with 3.5% pectin/hemoglobin solution and then the total mass of hemoglobin (flow rate×time×hemoglobin concentration). 3. Using the prepared stock solution, prepare hemoglobin/oligochitosan solutions at the following concentrations: 0.1 mg/mL, 0.05 mg/mL, 0.01 mg/mL, 0.005 mg/mL, 0.001 mg/mL. 4. Measure the absorbance of each concentration sample using a spectrometer at 410 nm. 5. On a computer, use a spreadsheet to plot the absorbance vs. concentration at 410 nm. Use the regression function to develop a linear equation relating concentration and absorbance with a y-intercept of zero. 6. Perform encapsulation with 3.5% low methoxyl pectin solution with hemoglobin. 7. Filter the sample through a 0.45 μm pore-size microfilter to collect the supernatant. 8. Using a micropipette, transfer 1 mL of the supernatant into a cuvette. 9. Use the spectrometer to obtain the absorbance of the supernatant at 410 nm. 10. Using the linear regression obtained from the standard curve in the spreadsheet, calculate the hemoglobin concentration of the supernatant and then determine the unencapsulated mass (hemoglobin concentration×total supernatant volume). 11. Calculate the encapsulated percentage using Equation 1.

Encapsulation %=[Total Mass−Unencapsulated Mass]/Total Mass×100 (Equation 1)

Data Analysis for Encapsulation:

An inverted microscope accompanied with an image processing program was used to analyze the data. The inverted microscope took a picture of the capsules in the solution and provided a scale bar relative to the magnification of the microscope. The images were then inserted into the ImageJ program which allowed for accurate measurement by determining the distance per pixel ratio. The scale bar in the picture was used to create a standard and then the distance was measured. The distance at which the largest diameter of the capsules occurred was measured to ensure consistency of measurements. Measurements of 100 capsules were averaged for input into Design Expert®. The software then modeled parameter effects on diameter, and performed optimization.

Statistical Methodology for Encapsulation: When the linear regression was generated, the “y” intercept was manually set to zero. By ignoring the intercept in the equation, it assumed a perfectly linear standard curve and ignored deviation. The flow rate of the encapsulator was influenced by solution composition and potential blockages due to high viscosities. Inconsistent flow rates resulted in experimental error when calculating the encapsulated percentage.

### Methods for Size Studies

Solution Preparation:

The following steps were performed: 1. Prepare pectin: viscosity enhancer: solution modifier solution 2. Use Nanopure water to prepare 50 mL of each of the solutions using: 4.07% low methoxyl pectin, 4% viscosity enhancer, and 5% solution modifier. 3. Combine 47.5 mL of the pectin solution and 2.5 mL of the viscosity enhancer solution, mix thoroughly using a stir plate with a stir bar. Allow to mix for at least 10 minutes. 4. Mix 35 mL of the pectin: viscosity enhancer solution with 7.5 mL of the solution modifier solution and 7.5 mL of nanopure water. Use a stir plate and a stir bar to ensure the solution is mixed thoroughly. Allow to mix for at least 10 minutes. Oligochitosan solution: 1. Prepare oligochitosan solution 2. Prepare a 5% oligochitosan solution using 2 kD oligochitosan and nanopure water.

Syringe Preparation:

The following steps were performed: 1. Take a 2.5 mL syringe and carefully remove and properly dispose of the needle tip. 2. Add approximately 1.5 to 2 mL of the pectin: viscosity enhancer: solution modifier solution by putting the syringe in the middle of the solution and slowly pulling up on the syringe. Ensure that there are no air bubbles in the solution, as this can cause problems during the vibration electrospraying process. 3. Attach a 30-gauge needle to the end of the syringe. 4. Take a piece of aluminum foil that is approximately 1 by 2 inches. Fold the aluminum foil in half so that it is 0.5 by 2 inches. Wrap the piece of the aluminum foil around the needle tip but be sure that the end of the needle tip is still exposed. 5. Use electrical tape to secure the aluminum foil to the syringe but be sure to keep a large enough portion of the aluminum foil exposed so that a clamp can be attached to it.

Electrospinning Device Set Up:

The following steps were performed: 1. Connect all yellow/green cords to the grounding strip on the side of the hood. 2. Place the regulator within the hood and connect to the generator. 3. Ensure that the power cord for the generator is attached and plugged in. 4. Use duct tape to attach the two-part emergency magnetic shut off to the hood ledge and the hood sash to complete the circuit of the generator. 5. Secure the syringe pump in between two clamps on a ring stand. The height of the clamps can be adjusted to the desired distance between the syringe needle and the collection oligochitosan solution. 6. Place the syringe into the syringe pump. Move the pusher plate down so that it is touching the end of the syringe. The plate can be moved slightly by using the small crank at the bottom of the syringe pump. 7. Take the red clamp that is connected to the regulator box and attach it to the aluminum foil on the syringe. 8. Place a petri dish with approximately 20 mL of the oligochitosan solution below the syringe needle. 9. Place the end of solution grounding wire into the petri dish with the oligochitosan solution.

Data Analysis:

An inverted microscope accompanied with an image processing program was used to analyze the data. The inverted microscope took a picture of the capsules in the solution and provided a scale bar relative to the magnification of the microscope. The images were then inserted into the ImageJ program which allowed for accurate measurement by determining the distance per pixel ratio. The scale bar in the picture was used to create a standard and then the distance was measured. The distance at which the largest diameter of the capsules occurred was measured to ensure consistency of measurements. Measurements of 100 capsules were averaged for input into Design Expert®. The software then modeled parameter effects on diameter, and performed optimization.

Statistical Methodology:

For each trial, 100 or more capsules were measured for the data to be statistically significant. The average along with the standard deviation of each trial was then calculated using a spreadsheet.

### In Vitro Stability Methods

Calcium chloride or glutaraldehyde solution preparation: The following steps were performed: 1. Prepare 100 mM calcium chloride stock solution. 2. Dilute the stock calcium chloride stock solution to 10 mM, 25 mM and 60 mM concentrations. Glutaraldehyde solutions included 0.75 M, 0.5 M and 0.1 M.

Plasma Buffer Preparation:

The following steps were performed: 1. Add 700 mL of nanopure water to 1 L volumetric flask. 2. Add various reagents. i. 8.035 g of Sodium Chloride ii. 0.355 g of Sodium Bicarbonate iii. 0.225 g of Potassium Chloride iv. 0.231 g of Potassium Phosphate Dibasic Trihydrate v. 0.311 g of Magnesium Chloride Hexahydrate vi. 39 mL of 1 M Hydrochloric Acid vii. 0.292 g of Calcium Chloride viii. 0.072 g of Sodium Sulfate ix. 6.118 g of Tris(hydroxymethyl) Aminomethane. 3. Add Nanopure to 1 L line in volumetric flask. 4. Adjust pH to 7.4±0.05 using 1 M Hydrochloric Acid i. Adjust before each use.

Stability Protocol:

The following steps were performed: 1. Produce hydrogel carriers. 2. Combine trials into a 50 mL conical tube. 3. Photograph combined samples. 4. Centrifuge the combined trials at the following parameters. i. Speed: 1000 rpm ii. Time: 3 minutes iii. Acceleration: 9 iv. Deceleration: 5 v. Temp: 20° C. 5. Aspirate off the majority of the supernatant (oligochitosan) and discard. 6. Aspirate off remaining pellet and add 20 mL of nanopure water. 7. Suspend pellet in nanopure. 8. Photograph samples. 9. Centrifuge at the following parameters. i. Speed: 1000 rpm ii. Time: 3 minutes iii. Acceleration: 9 iv. Deceleration: 5 v. Temp: 20° C. 10. Aspirate off the majority of the supernatant (nanopure) and discard. 11. Aspirate off remaining pellet and add 20 mL of plasma buffer. 12. Suspend pellet. 13. Photograph sample. 14. Store capsules and photograph for two weeks.

### Results

Encapsulation Efficiency:

First, the total mass of hemoglobin in the prepared solution was determined. The flow rate was determined with a sequence of time trials where the volume extruded by the electrospray device was measured for a specific amount of time. The average volume per time was calculated for a flow rate of 0.00022 mL/s. A stock concentration of 1 mg/mL hemoglobin was prepared for hemoglobin encapsulation with pectin and oligochitosan. The stock hemoglobin solution was extruded for 15 min with the encapsulator. Using this data, Equation 2 was used to determine the total mass of hemoglobin in the stock solution as 0.2 mg.

Total Hemoglobin Mass=Flow Rate×Time×Hemoglobin Concentration (Equation 2)

After the stock solution was extruded through the electrospray device for 15 min to form pectin/oligochitosan capsules containing hemoglobin, a sample of the supernatant was obtained. The absorbance at 410 nm of the supernatant was measured in a spectrophotometer. Previously, the spectrophotometer was used to develop a standard curve with known hemoglobin concentrations at 410 nm. The standard curve yielded Equation 3:

Absorbance=5.51×Hemoglobin Concentration (Equation 3)

The sample had an absorbance of 0.001 at 410 nm as well as a sample volume of 15 mL. Equation 3 was used to determine the concentration of hemoglobin in the supernatant of 1.81*10⁻⁴ mg/mL. By multiplying the concentration of excess hemoglobin by the initial volume of 15 mL, the mass of unencapsulated hemoglobin to be 0.00272 mg was determined. Equation 1 uses the mass of the initial, total hemoglobin in the stock solution and the mass of unencapsulated hemoglobin to determine the encapsulation efficiency.

The encapsulation efficiency of hemoglobin with the pectin/oligochitosan capsules produced on an encapsulator device was determined to be 98.6%.

An encapsulation efficiency of 97.3±3.6% proves promising for the oxygen carrying capacity of the capsules. This shows the encapsulation method can allow for a significant amount of the hemoglobin to be contained within the capsules without the use of additional methods to insert the hemoglobin into the capsules.

Size Reduction:

Two sets of parameter tests were performed. The results of the first and second experiments are shown in Tables 1 and 2, respectively.

TABLE 1

| Run | Voltage (V) | Flow Rate (ml/h) | Height (cm) | Diameter (um) |
|---|---|---|---|---|
| 1 | 10 | 2.415 | 12.5 | 12.37 |
| 2 | 20 | 1.38 | 25 | 7.47 |
| 3 | 20 | 3.45 | 18.75 | 11.60 |
| 4 | 30 | 3.45 | 18.75 | 10.99 |
| 5 | 10 | 2.415 | 25 | No Data |
| 6 | 20 | 1.38 | 12.5 | 11.92 |
| 7 | 30 | 2.415 | 25 | 11.48 |
| 8 | 30 | 2.415 | 12.5 | 12.89 |
| 9 | 20 | 3.45 | 12.5 | 17.80 |
| 10 | 20 | 2.415 | 18.75 | 10.90 |
| 11 | 30 | 1.38 | 18.75 | 11.39 |
| 12 | 20 | 2.415 | 18.75 | 11.05 |
| 13 | 20 | 2.415 | 18.75 | 9.86 |
| 14 | 20 | 2.415 | 18.75 | 10.06 |
| 15 | 10 | 3.45 | 18.75 | 8.35 |
| 16 | 10 | 3.45 | 18.75 | 8.01 |
| 17 | 20 | 3.45 | 25 | 15.91 |

Table 1 summarizes the results of the first parameter test. The initial test was incomplete; capsules were difficult to collect at the 25 cm height. Additional variabilities in the test included: placement of the petri dish, placement of the grounding wire, and side stream formation. In an attempt to reduce or eliminate the effects of these variabilities, placement of the dish and wire were standardized, and the maximum height was reduced.

TABLE 2

| Run | Voltage (kV) | Flow Rate (ml/h) | Height (cm) | Diameter (um) |
|---|---|---|---|---|
| 1 | 17 | 0.69 | 18 | 7.85 |
| 2 | 17 | 1.17 | 22 | 7.86 |
| 3 | 17 | 0.69 | 18 | 7.32 |
| 4 | 17 | 0.69 | 18 | 7.44 |
| 5 | 22 | 1.17 | 18 | 7.61 |
| 6 | 17 | 0.69 | 18 | 6.76 |
| 7 | 17 | 0.21 | 22 | 6.62 |
| 8 | 12 | 0.69 | 14 | 6.97 |
| 9 | 17 | 0.69 | 18 | 6.98 |
| 10 | 12 | 0.69 | 22 | 6.42 |
| 11 | 12 | 1.17 | 18 | 7.18 |
| 12 | 22 | 0.69 | 14 | 10.94 |
| 13 | 22 | 0.69 | 22 | 8.58 |
| 14 | 17 | 1.17 | 14 | 11.96 |
| 15 | 12 | 0.21 | 18 | 6.57 |
| 16 | 17 | 0.21 | 14 | 8.85 |
| 17 | 22 | 0.21 | 18 | 6.92 |

This data was used to create a quadratic predictive model in Design Expert®. The model was reduced by removing insignificant terms one at a time, beginning with the term having the highest p-value. The resulting relationship between the parameters and capsule diameter is shown in Equation 4 below:

Diameter=7.19A+0.71B−1.16C+1.35C² (Equation 4)

Where A, B, and C represent voltage, flow rate, and height, respectively. Design Expert® also provided an ANOVA table for the model:

TABLE 3

| | Sum of | Degrees of | | | |
|---|---|---|---|---|---|
| Source | Squares | Freedom | Mean Square | F Value | p-value |
| Model | 28.36 | 4 | 7.09 | 8.96 | 0.0014 |
| A: Voltage | 5.78 | 1 | 5.78 | 7.30 | 0.0192 |
| B: Flow Rate | 4.06 | 1 | 4.06 | 5.13 | 0.0428 |
| C: Height | 10.81 | 1 | 10.81 | 13.66 | 0.0031 |
| C² | 7.70 | 1 | 7.70 | 9.74 | 0.0089 |

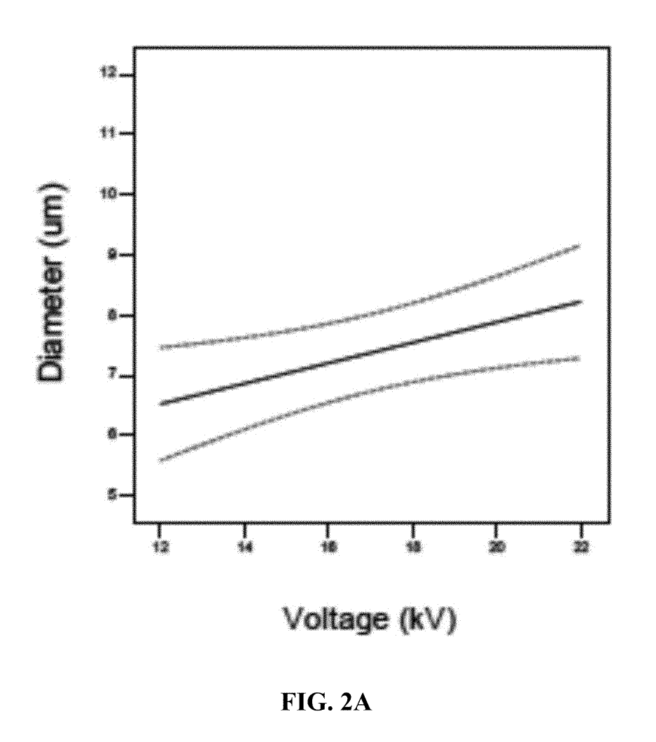

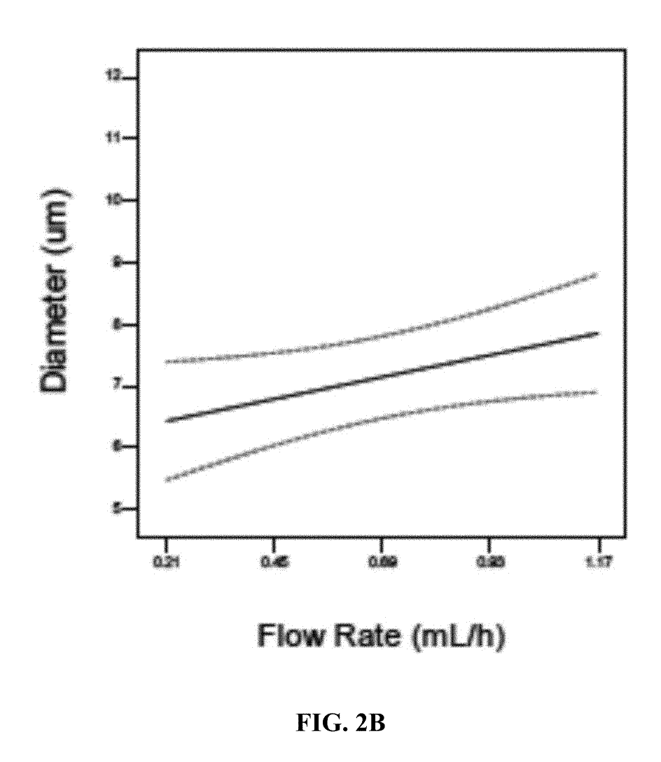

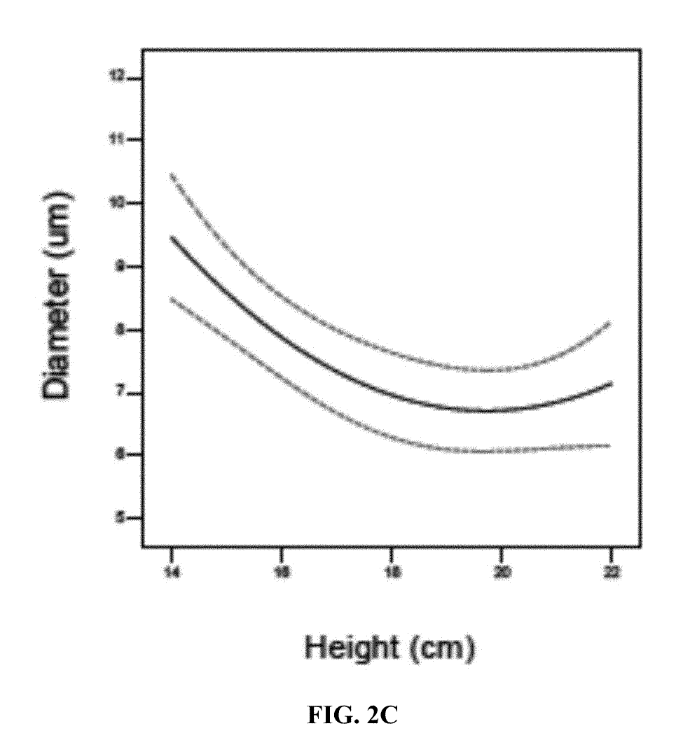

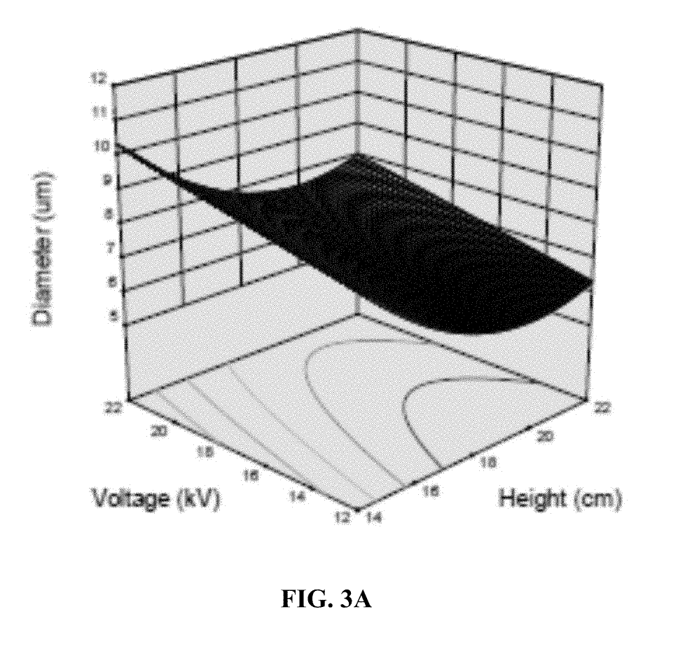

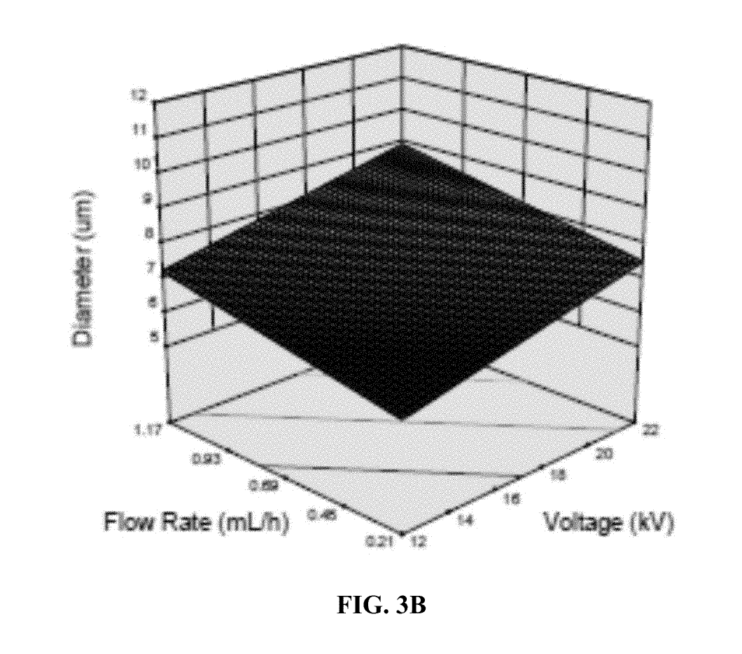

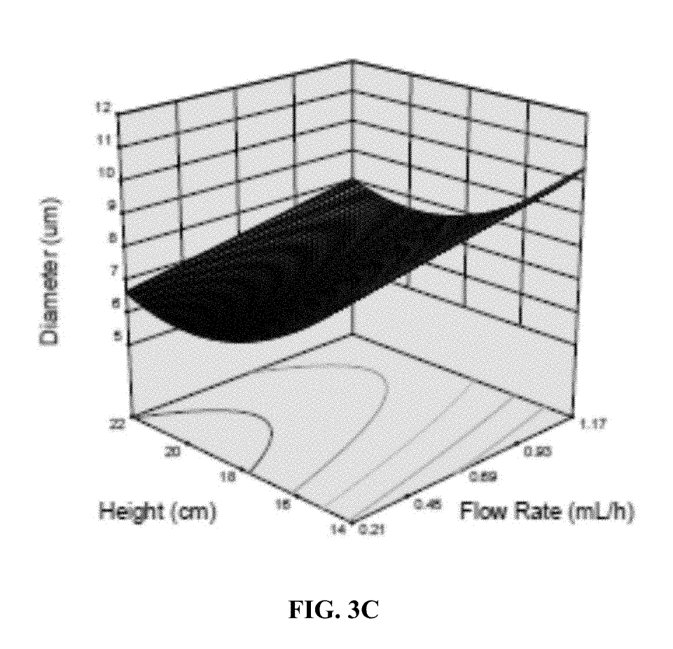

From Table 3, it can be seen that all model terms are significant, as the p-value for each term is less than 0.05. In context, this means that each parameter has a significant effect on capsule diameter, and the height of the syringe effect is parabolic. Single-variable trends and multivariate effects can be seen in FIGS. 2 and 3, respectively.

The voltage and flow rate seem to be positively correlated with diameter, increasing either parameter results in an increase in diameter. This is slightly confounding, as the art suggests voltage to be a dispersion source of fluid microdroplets, and increasing that force would increase the number of droplets, thus decreasing their size. In addition, diameter appears to have a parabolic trend when compared to height alone.

The surface response curves in FIG. 3 show the two-variable relationships between parameters. None of the interactions between parameters were statistically significant, so these trends are projections of the single-variable relationships in FIG. 2.

The final statistical analysis performed for this experiment was a summary model in RStudio. The same linear and quadratic terms were used as a means to verify the Design Expert® model calculations. Salient statistical values are summarized in Table 4.

TABLE 4

| Parameter | Coefficient | p-value | Correlation |
|---|---|---|---|
| Voltage | 0.1727 | 0.0052 | 0.1635 |
| Flow Rate | 0.2020 | 0.0111 | 0.1339 |
| Height | −3.319 | 0.0002 | −0.2188 |
| H² | 0.0839 | 0.0002 | −0.2032 |

Table 4 agrees with the Design Expert® quadratic predictive model in that each term has a significant effect on the diameter, as all p-values are less than 0.05. This table does yield new statistical information, the correlation for each term. The absolute value of the correlation is an approximate representation of how much variability in the response variable is accounted for by each input variable. The sum of the correlations here is about 0.7, indicating that about 30 percent of diameter variation is unaccounted for. It should be noted that Design Expert® showed a significant lack of fit in the quadratic model, however, other common models such as linear, two-factor interaction, and cubic polynomials had no improvements in fit. The statistical significance of each term implies that there is a quadratic relationship, and the lack of fit could be explained by the 30 percent of unrepresented variation in diameter.

The size reduction was achieved as illustrated by FIG. 4 by altering the parameters on an electrospray device. The parameters, voltage, height, and flow rate of the electrospray device were altered in order to consistently achieve an average capsule diameter of 7-10 microns.

Design Expert®, a statistical analysis program, generated a list of trials in which parameters were varied while staying within a user-designated range constrained by device limitations. The trials were conducted on the electrospray device using unvaried solution composition to reduce experimental error. The average diameter for each trial was obtained using ImageJ software to measure the capsule size captured on an inverted, optical microscope. The resulting average diameters were inputted into Design Expert® to analyze the impact of each parameter on average capsule diameter. A secondary statistical analysis conducted using RStudio confirmed that parameters' statistical influence on the capsule diameter accounted for approximately 70% of the resulting size, thus indicating the investigated parameters are a main determinant in capsule size. A quadratic predictive model generated in Design Expert® further indicates the relationship between the parameters and capsule diameter. The quadratic model was modified to eliminate unrelated terms, resulting in the best fit for the data. From the adjusted quadratic model, the calculated parameters to produce capsules with an average diameter of 7 microns on an electrospray device from 3.25% pectin/oligochitosan solution are: voltage of 13.5 kV, flow rate of 1.1 mL/h, and height of 18.8 cm.

Stability:

In order for the pectin/oligochitosan capsules to provide a competitive alternative to traditional blood donations, the oxygen therapeutic prototypes must have a shelf life comparable to natural red blood cells at 42 days. After successfully reducing the size of the capsules, the initial stability of the produced capsules was investigated.

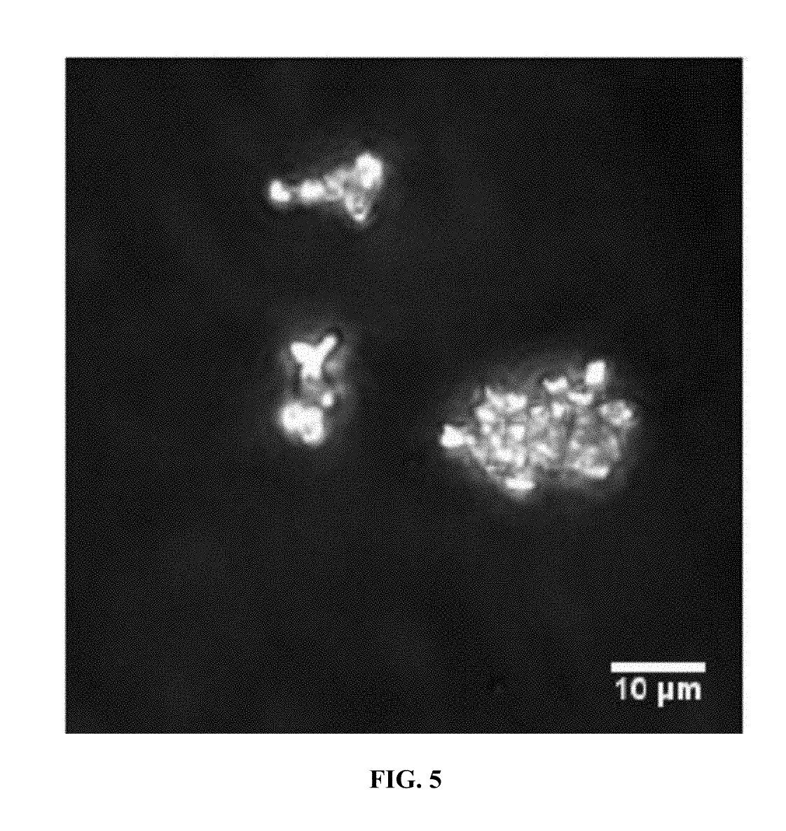

Previous stability of larger capsules was achieved with a treatment of 150 mM CaCl₂ solution. However, due to the size reduction of the capsules, it was determined that 150 mM was too great of a concentration. FIG. 5 indicates the collapsed capsules after treatment with 150 mM CaCl₂.

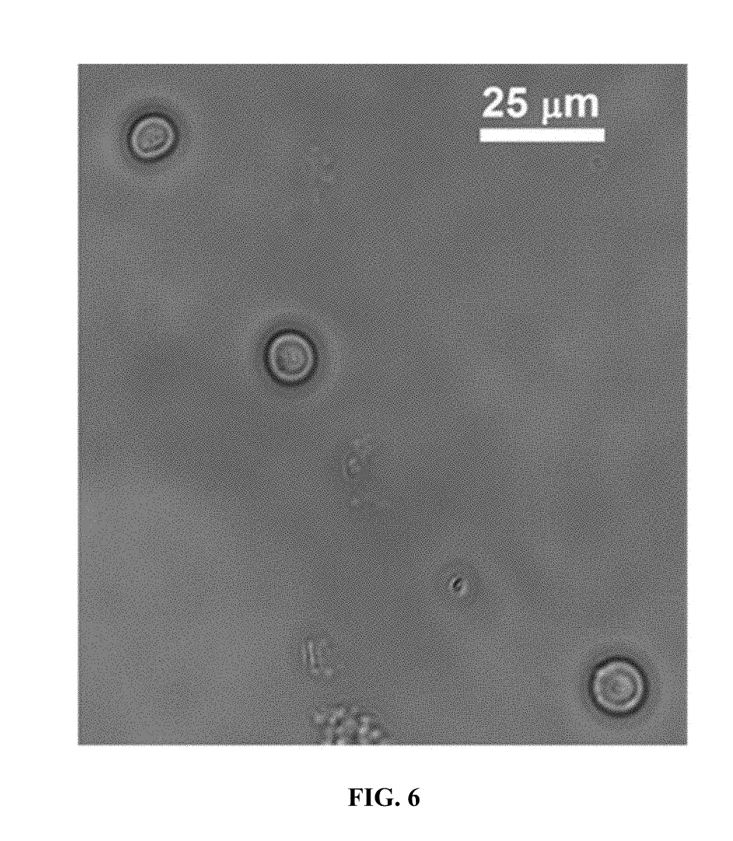

The excess of calcium may facilitate unnecessary binding between the pectin and oligochitosan polymers, compromising the morphology of the capsule. After the initial stability testing with 150 mM calcium solution, it was concluded that the concentration must be modified in the stability treatment protocol to accommodate the decreased capsule size. A range from 10 mM-100 mM calcium solutions were prepared in order to improve the stability treatment protocol. The capsules were treated with 10 mM, 25 mM, 60 mM, and 100 mM calcium solutions, then incubated for 30 minutes. After incubation, the capsules were washed with nanopure water, isolated through centrifugation, then transferred into a plasma stability buffer for long term storage. In addition, another set of capsules were treated with 0.1 M glutaraldehyde and exposed to the plasma buffer. As seen in FIG. 6 the glutaraldehyde treatment maintained the size and the shape of the capsules.

It was concluded that the lower concentration of 10 mM and 25 mM calcium solutions showed the fewest number of collapsed capsules (of the calcium exposed capsules) after incubation. The trial treated with 10 mM calcium solution was transferred into a plasma buffer, mimicking human blood plasma, for long term storage. The capsules were then visualized each week, until capsule degradation rendered the majority of stored capsules unrecognizable from the initial size and morphology images were taken immediately after incubation in 10 mM calcium solution.

The tested range of stability solution indicated a concentration of 10 mM calcium decreased the number of collapsed capsules after a 30-minute incubation period. While decreasing the stability solution concentration to 10 mM calcium solution prevented collapsing, it did not prolong the long term stability in plasma buffer. While results show a concentration of 10 mM calcium solution improved studies, it may not be optimal. Further, treatment of capsules with 0.1 M glutaraldehyde (without CaCl₂) showed good results for stability in plasma buffer.

Accordingly, hydrogel microcapsules with an approximate size of 6 to 8 microns were produced through the use of an electrospraying technique on an electrospinner. The varying electrospinner parameters including voltage, flow rate, and distance between the needle tip and the collection solution were tested to determine the specifications needed to produce the desired shape and size. The calculated parameters to produce capsules with an average diameter of seven microns on an electrospray device from 3.25% pectin/oligochitosan solution are: voltage of 13.5 kV, flow rate of 1.1 mL/h, and height of 18.8 cm. The hydrogels were determined to have a 96% encapsulation efficiency, suggesting the capsules are effectively able to encapsulate hemoglobin during the production process and that the hydrogel is stable enough to contain hemoglobin. Stability testing was also performed to increase the shelf life of the capsules. It was determined that the hydrogel capsules were more stable at lower concentrations of the stability solution of calcium chloride. Of the concentrations tested, the 10 mM calcium chloride solution proved to be most effective in avoiding adverse effects of treatment. In addition, it was determined that 0.1 M glutaraldehyde could maintain the shape and size of capsules in stability studies, thereby providing a potential alternative to calcium chloride treatment.

What is claimed is:

1. A synthetic particle comprising:
pectin; and
oligochitosan,
wherein the synthetic particle has a biconcave discoid shape, and a largest linear dimension of about 4 μm to about 12 μm.

2. The synthetic particle of claim 1, wherein pectin is present at about 5% to about 30% by weight.

3. The synthetic particle of claim 1, wherein oligochitosan is present at about 1% to about 5% by weight.

4. The synthetic particle of claim 1, further comprising a divalent cation, a covalent cross-linking agent or a combination thereof.

5. The synthetic particle of claim 1, wherein pectin is low methoxy pectin.

6. The synthetic particle of claim 1, wherein the oligochitosan has a molecular weight of about 1 kD to about 5 kD.

7. The synthetic particle of claim 1, further comprising a bioactive agent.

8. The synthetic particle of claim 7, wherein the bioactive agent is present at about 0.1% to about 3% by weight.

9. The synthetic particle of claim 7, wherein the bioactive agent is selected from the group consisting of a therapeutic agent, an imaging agent and a combination thereof.

10. The synthetic particle of claim 7, wherein the bioactive agent is hemoglobin.

11. The synthetic particle of claim 1, wherein the synthetic particle is a hydrogel.

12. The synthetic particle of claim 1, wherein the synthetic particle has a volume of about 20 μm³ to about 315 μm³.

13. The synthetic particle of claim 1, wherein the synthetic particle has a surface area of about 45 μm² to about 300 μm².

14. A method of making a synthetic particle having a shape of a red blood cell, the method comprising:
electrospraying a pectin solution comprising pectin, a viscosity enhancer, a solution modifier and a first solvent into an oligochitosan solution comprising oligochitosan and a second solvent to provide a particle suspension comprising the synthetic particle of claim 1.

15. The method of claim 14, wherein the pectin solution further comprises a bioactive agent.

16. The method of claim 14, further comprising adding a solution that includes a divalent cation to the particle suspension, wherein the divalent cation is present in the solution at a concentration of about 10 mM to about 150 mM.

17. The method of claim 14, further comprising adding a solution that includes a covalent cross-linking agent to the particle suspension, wherein the covalent cross-linking agent is present in the solution at a concentration of about 0.1 M to about 0.75 M.

18. The method of claim 14, wherein electrospraying is performed at a voltage of about 5 kV to about 30 kV.

19. The method of claim 14, wherein the pectin solution is electrosprayed at a height of about 5 cm to about 25 cm above the oligochitosan solution and/or at a flow rate of about 0.21 mL/h to about 15 mL/h.

20. The method of claim 14, wherein the viscosity modifier is present in the pectin solution at about 2% to about 5% by weight/volume and/or the solution modifier is present in the pectin solution at about 1% to about 30% by weight/volume.
